(19) United States
(12) Patent Application Publication
(10) Pub. No.: US 2012/0165553 A1
(43) Pub. Date: Jun. 28, 2012

(54) IMMUNOSTIMULANT

(75) Inventors: Kenji Ogawa, Saitama (JP); Takeshi Nakano, Saitama (JP)

(73) Assignee: RIKEN, Wako-shi (JP)

(21) Appl. No.: 13/323,690

(22) Filed: Dec. 12, 2011

(30) Foreign Application Priority Data
Dec. 10, 2010 (JP) 2010-276017

(51) Int. Cl. C07D 313/14; C07C 49/513; C07J 9/00

(52) U.S. Cl. 549/268; 552/541; 568/369

(57) ABSTRACT

[Problem to be Solved]

An immunostimulant highly safe and administrable for a long period of time as well as a pharmaceutical composition containing the immunostimulant as an active ingredient and drink or food products for immunostimulation are developed and provided.

[Solution]

An immunostimulant containing a compound represented by the following general formula (I) or a pharmaceutically acceptable salt thereof as an active ingredient is provided.

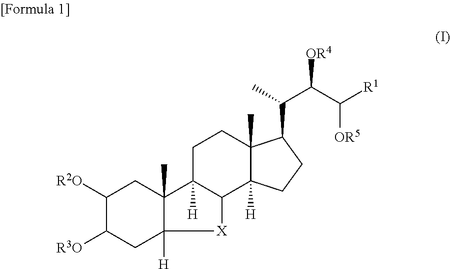

R¹ is a hydrogen atom or a substituted or unsubstituted alkyl group having 1 to 6 carbon atoms, R² to R⁵ are independently a hydrogen atom, or a substituted or unsubstituted lower alkyl or lower acyl group, X is —(CH₂)₂—, —(CH₂)₃—, —(CH₂)₄—, —CO—CH₂—, —CO—(CH₂)₂—, —CO—(CH₂)₃—, —CH₂—CO—CH₂—, —CO—O—CH₂—, —CO—O—(CH₂)₂—, —O—CO—CH₂—, —O—CO—(CH₂)₂—, —CH₂—O—CH₂—, —CH₂—O—(CH₂)₂—, —O—CH₂—, —O—(CH₂)₂—, —O—(CH₂)₃—, —NHY—CO—CH₂—, —NHYCO—(CH₂)₂—, —CO—NHY—CH₂— or —CO—NHY—(CH₂)₂—, Y herein is a hydrogen atom or a lower alkyl group.

### TECHNICAL FIELD

The present invention relates to an immunostimulant containing brassinosteroid as an active ingredient and a pharmaceutical composition and drink and food products containing the same.

### BACKGROUND ART

The immunostimulant has an action to enhance preventive and/or therapeutic effects in a host against infectious disease, etc., by activating the host immunity. For example, it is known that the immunostimulant enhances the innate immune system by promoting the cytokine production in a host or enhances the acquired immune system by promoting the phagocytosis and antigen presentation ability (Non Patent Literature 1).

Conventionally, known immunostimulants include components of bacteria represented by BCG or killed bacteria or those described in Patent Literatures 1 to 5, and the like.

### CITATION LIST

### Patent Literature

[Patent Literature 1] JP Patent Publication (Kokai) No. 5-213765 A (1993)
[Patent Literature 2] JP Patent Publication (Kokai) No. 2006-104068 A
[Patent Literature 3] JP Patent Publication (Kokai) No. 2009-114122 A
[Patent Literature 4] JP Patent Publication (Kokai) No. 2009-149523 A
[Patent Literature 5] JP Patent Publication (Kokai) No. 2010-235544 A

### Non Patent Literature

[Non Patent Literature 1] Janeway C A, Jr. et al., 2005, Immunobiology. 6th ed., GarlandScience

### SUMMARY OF INVENTION

### Technical Problem

An object of the present invention is to develop and provide an immunostimulant highly safe and administrable for a long period of time as well as a pharmaceutical composition and drink or food products for immunostimulation containing the immunostimulant as an active ingredient.

### Solution to Problem

To solve the above problem, the present inventors searched for a substance having the above-mentioned immunostimulating action from compounds commonly found in different plants including vegetables and fruits to begin with. As a result, the inventors have found that brassinosteroid in a very small amount has the immunostimulating action on an animal.

Brassinosteroid is a physiologically active substance distinctively found in plants which plays an essential role for plant development, chloroplast control, seed formation, etc. (Azpiroz R. et al., 1988, Plant Cell, 10:219-230; Clouse S. & Sasse J., 1998, Annu. Rev. Plant Physiol. Plant Mol. Biol., 49: 427-45; Mandava N., 1988, Annu. Rev. Plant Physiol. Plant Mol. Biol., 39:23-52; Sakurai A. et al., 1999, Brassinosteroids, Steroidal Plant Hormones, Tokyo: Springer). Brassinosteroid is also called a plant hormone owing to its steroid skeleton but exhibits different properties from animal steroid hormones because of its distinct extra-hydroxylated structure.

Known plant-derived compounds are, as isoflavone in soybean, substances which bond with a steroid hormone receptor due to the analogy in terms of three dimensional structure and imitate the action. Isoflavone is widely known to be efficacious for health improvement but also reported likely to cause side effects such as miscarriage, when taken in a large amount by a pregnant woman, or the like (Food Safety Commission, May 2006, “Basic approaches to evaluating the safety of Food for Specified Health Uses containing soy isoflavone (in Japanese)”). On the other hand, brassinosteroid does not activate a steroid hormone receptor, thus being free from such side effects. Also, most of the plant-derived compounds efficacious in the human body such as crude drug ingredients are usually secondary metabolite compounds in a plant physiological sense and do not play an important role at all for the plant growth cycle; however, brassinosteroid is a physiologically active substance playing an important role for the plant growth cycle even in plants and commonly present in many different plants (Shokubutsu Horumon Handobukku (in Japanese), (Plant Hormone Handbook) “Brassinosteroid”, edited by Nobutaka Takahashi, Yoshio Masuda, Baifukan, 1994, p. 203-240). For this reason, brassinosteroid contained in vegetables, etc., consumed daily by human is considered to be free of or have extremely scarce side effects from the consumption, and is hence a highly safe substance to the human body. The present invention, based on the above novel findings, utilizes such a brassinosteroid as an immunostimulant and provide as follows.

(1) An immunostimulant comprising as an active ingredient a compound represented by the following general formula (I) or a pharmaceutically acceptable salt thereof:

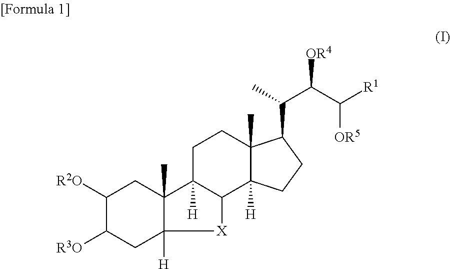

wherein,

R¹ is a hydrogen atom or a substituted or unsubstituted alkyl group having 1 to 6 carbon atoms,

R² to R⁵ are independently a hydrogen atom, or a substituted or unsubstituted lower alkyl or lower acyl group, and

X is —(CH₂)₂—, —(CH₂)₃—, —(CH₂)₄—, —CO—CH₂—, —CO—(CH₂)₂—, —CO—(CH₂)₃—, —CH₂—CO—CH₂—, —CO—O—CH₂—, —CO—O—(CH₂)₂—, —O—CO—CH₂—, —O—CO—(CH₂)₂—, —CH₂—O—CH₂—, —CH₂—O—(CH₂)₂—, —O—CH₂—, —O—(CH₂)₂—, —O—(CH₂)₃—, —NHY—CO—CH₂—, —NHYCO—(CH₂)₂—, —CO—NHY—CH₂— or —CO—NHY—(CH₂)₂—,

where Y is a hydrogen atom or a lower alkyl group.

(2) The immunostimulant according to (1), wherein R¹ is a 1,2-dimethylpropyl group.
(3) The immunostimulants according to (1) or (2), wherein R² to R⁵ are a hydrogen atom.
(4) The immunostimulant according to any one of (1) to (3), wherein X is —CO—O—CH₂—.
(5) The immunostimulant according to (4) represented by the following formula (II).

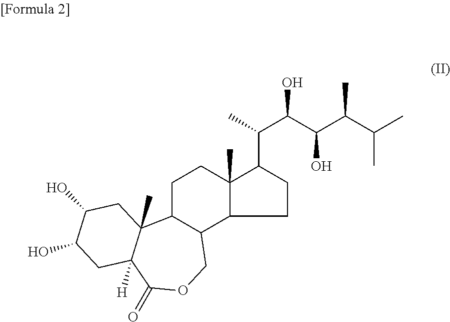

(6) A pharmaceutical composition comprising the immunostimulant according to any one of (1) to (5) as an active ingredient.
(7) A food or drink product for immunostimulation comprising the immunostimulant according to any one of (1) to (5) as an active ingredient.

The contents described in the specification and/or drawings of Japanese Patent Application No. 2010-276017, which is the basis of priority of the present application, are herein incorporated.

### Advantageous Effects of Invention

According to the present invention, an immunostimulant highly safe and administrable for a long period of time, a pharmaceutical composition and drink and food products for immunostimulation containing the immunostimulant as an active ingredient can be provided.

### BRIEF DESCRIPTION OF DRAWINGS

FIG. 1 shows the cell proliferation activity of mouse spleen cells (T cells and B cells) by brassinolide (BL).

FIG. 2 shows the activated antibody production against ovalbumin (OVA) antigen stimulation by brassinolide (BL).

FIG. 3 shows the effect of brassinolide (BL) on interferon-α gene expression in macrophages.

FIG. 4 shows the effect of brassinolide (BL) on interferon-β gene expression in macrophages.

FIG. 5 shows the effect of brassinolide (BL) on IL-12 subunit p35 gene expression in macrophages.

FIG. 6 shows the effect of brassinolide (BL) on IL-12 subunit p40 gene expression in macrophages.

FIG. 7 shows the effect of brassinolide (BL) on TNF-α gene expression in macrophages.

### DESCRIPTION OF EMBODIMENTS

### 1. Immunostimulant

The first embodiment of the present invention is an immunostimulant.

In the present invention, the “immunostimulant” refers to the drug which activates immunological effects in the living body of an animal. The immunity can be roughly classified into the innate immune system which is the non-specific defense via macrophages, neutrophils, NK cells, etc., and the acquired immune system which is the specific defense active on a specific target via T cells and B cells; however, the immunostimulant of the present invention is capable of activating both the innate immune system and acquired immune system.

The immunostimulant of the present invention comprises as an active ingredient at least one compound represented by the following general formula (I) or a pharmaceutically acceptable salt thereof.

### 1-1. Compound Represented by the General Formula (I)

The compound which is the active ingredient of the immunostimulant of the present invention and represented by the following general formula (I) is brassinosteroid (hereinafter referred to as “BR”). As described earlier, BR is a physiologically active substance which is biosynthesized in a living plant and involved in the plant growth regulation, photomorphogenesis, regulation of vascular bundle development, regulation of chloroplast function, seed formation, and the like. However, the BR of the present invention is not limited to the plant-derived BR biosynthesized in a living plant and extracted from the plant insofar as it has the structure represented by the following general formula (I) and may be those chemically synthesized.

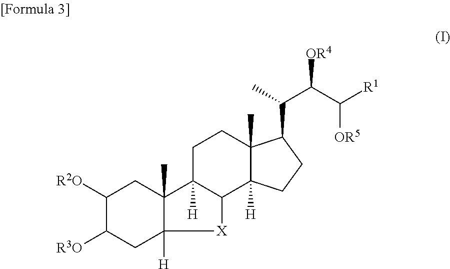

In the above general formula (I), R¹ is a hydrogen atom or a substituted or unsubstituted alkyl group having 1 to 6 carbon atoms. The carbon chain may be linear or branched when the number of carbon atoms is 3 to 6. Examples of the alkyl group having 1 to 6 carbon atoms include a methyl group, an ethyl group, a propyl group, an isopropyl group, a tert-butyl group, 2-methylpropyl group, a 1,2-dimethylpropyl group and a 1-ethyl-2-methylpropyl group. The 1,2-dimethylpropyl group is preferable.

The “substituted or unsubstituted” means that the alkyl group having 1 to 6 carbon atoms constituting R¹ and/or the lower alkyl group or the lower acyl group constituting R² to R⁵ to be described later may or may not be substituted with a substituent. Examples of the “substituent” used herein include halogen, a hydroxyl group, a lower alkyl group, a lower alkenyl group, a lower alkynyl group, a lower alkoxy group, an amino group, a lower alkylamino group, an amide group, an N-lower alkyl amide group, a cyano group, a nitro group, a carboxyl group, a carbamoyl group, a sulfate group, and a sulfite group. The halogen, the hydroxyl group, the lower alkyl group, the lower alkoxy group and the cyano group are preferable. The number of the substituent is one or more, for example, 1 to 4, 1 to 3 or 2 per compound.

R² to R⁵ are independently a hydrogen atom, or a substituted or unsubstituted lower alkyl or lower acyl group.

In the present specification, the “lower alkyl group” is a linear alkyl group having 1 to 4 carbon atoms or a branched alkyl group having 3 to 4 carbon atoms and examples include a methyl group, an ethyl group, an n-propyl group, an iso-propyl group, an n-butyl group, an isobutyl group, and a tert-butyl group.

In the present specification, the “lower acyl group” is a linear acyl group having 1 to 6 carbon atoms or a branched acyl group having 3 to 4 carbon atoms and examples include a formyl group, an acetyl group, a propionyl group, an isopropionyl group, an n-butyryl group, an isobutyryl group, a sec-butyryl group, and a tert-butyryl group.

The “halogen” is a fluorine atom (F), a chlorine atom (Cl), a bromine atom (Br), or an iodine atom (I).

In the present specification, the “lower alkenyl group” is a linear or branched alkenyl group having 2 to 8 carbon atoms, preferably 2 to 4 carbon atoms. Specific examples include an ethenyl group, a propenyl group, a butenyl group, a butadienyl group, a pentenyl group, a pentadienyl group, a hexenyl group, a hexadienyl group, a heptenyl group, a heptadienyl group, an octenyl group, octadienyl group, and isomers thereof.

In the present specification, the “lower alkynyl group” is a linear or branched alkynyl group having 2 to 8 carbon atoms, preferably 2 to 4 carbon atoms. Specific examples include an ethynyl group, a propynyl group, a butynyl group, a butadiinyl group, a pentynyl group, a pentadiinyl group, a hexynyl group, a hexadiinyl group, a heptynyl group, heptadiinyl group, octynyl group, and octadiinyl group.

In the present specification, the “lower alkoxy group” is a linear or branched alkoxy group having 1 to 6 carbon atoms, preferably 1 to 4 carbon atoms. Specific examples include a methoxy group, an ethoxy group, an n-propoxy group, an isopropoxy group, an n-butoxy group, an isobutoxy group, an s-butoxy group, a t-butoxy group, an n-pentyloxy group, an isopentyloxy group, an n-hexyloxy group, and an isohexyloxy group.

In the present specification, the “lower alkylamino group” is a group formed with one or two of the above lower alkyl group substituents at the amino group.

In the present invention, R² to R⁵ are preferably a hydrogen atom.

Further, X in the above general formula (I) represents —(CH₂)₂—, —(CH₂)₃—, —(CH₂)₄—, —CO—CH₂—, —CO—(CH₂)₂—, —CO—(CH₂)₃—, —CH₂—CO—CH₂—, —CO—O—CH₂—, —CO—O—(CH₂)₂—, —O—CO—CH₂—, —O—CO—(CH₂)₂—, —CH₂—O—(CH₂)₂—, —O—CH₂—, —O—(CH₂)₂—, —O—(CH₂)₃—, —NHY—CO—CH₂—, —NHYCO—(CH₂)₂—, —CO—NHY—CH₂— or —CO—NHY—(CH₂)₂—, where Y is a hydrogen atom or a lower alkyl group.

In the present invention, X is preferably —CO—CH₂— or —CO—O—CH₂—. —CO—O—CH₂— is more preferable.

Particularly preferable BR as the active ingredient contained in the immunostimulant of the present invention is Brassinolide (hereinafter referred to as “BL”) biosynthesized in a living plant and represented by the following formula (II).

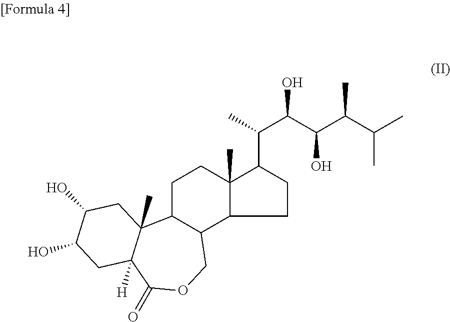

Further, examples of preferable BRs as the active ingredient contained in the immunostimulant of the present invention in addition to the above BL include compounds such as Castasterone represented by the following formula (III), iso-Brassinolide represented by the formula (IV), 6a-carba-Brassinolide represented by the formula (V), 5-epi-carba-Brassinolide represented by the formula (VI), iso-carba-Brassinolide represented by the formula (VII), homo-carba-homo-Brassinolide represented by the formula (VIII), and 6-deoxo-methylene-Castasterone represented by (IX).

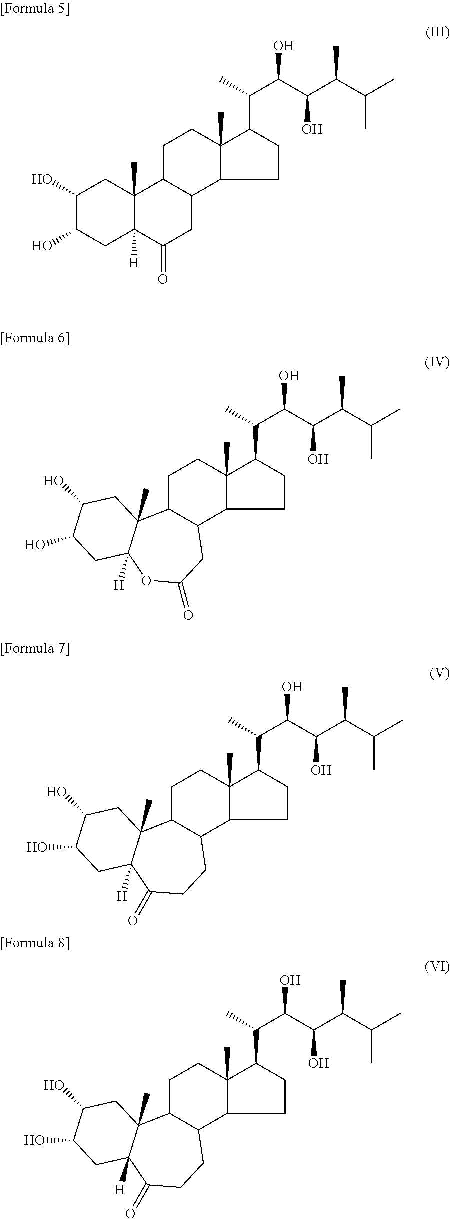

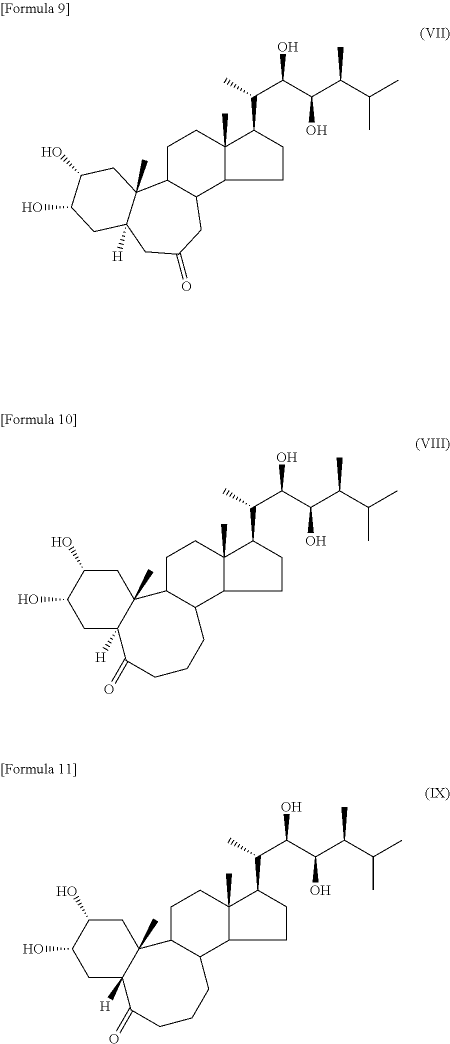

The immunostimulant of the present invention may contain single BR or two or more BRs. An example includes a mixture of the above BL and Castasterone.

### 1-2. Pharmaceutically Acceptable Salt

The “pharmaceutically acceptable salt” is a salt of the compound represented by the general formula (I), i.e., a salt of BR, which means a pharmaceutically nontoxic salt of an active compound prepared using a base or an acid based on a specific substituent (e.g., a hydroxyl group) of BR. The salt can be classified into base addition salt and acid addition salt based on the base or acid used.

Examples of the “base addition salt” include alkali metal salts such as a sodium salt and a potassium salts; alkaline earth metal salts such as a calcium salt and a magnesium salt; aliphatic amine salts such as a trimethylamine salt, a triethylamine salt, a dicyclohexylamine salt, an ethanolamine salt, a diethanolamine salt, a triethanolamine salt, and a procaine salt; aralkylamine salts such as N,N-dibenzylethylenediamine; heterocyclic aromatic amine salts such as a pyridine salt, a picoline salt, a quinoline salt, and an isoquinoline salt; basic amino acid salts such as an arginine salt and a lysine salt; quarternary ammonium salts such as a tetramethylammonium salt, a tetraethylammonium salt, a benzyltrimethylammonium salt, a benzyltriethylammonium salt, a benzyltributylammonium salt, a methyltrioctylammonium salt, a tetrabutylammonium salt, and an ammonium salt.

Examples of the “acid addition salt” include inorganic acid salts such as hydrochloride, sulphate, nitrate, phosphate, carbonate, bicarbonate, and perchlorate; organic acid salts such as acetate, propionate, lactate, maleate, fumarate, tartrate, malate, citrate, and ascorbate; sulfonates such as methanesulfonate, isethionate, benzenesulfonate, and p-toluenesulfonate; and acidic amino acid salts such as aspartate and glutamate.

### 1-3. Effects

The immunostimulant of the present embodiment contains BR as an active ingredient, which is contained in almost all plants, to begin with vegetables such as tomatoes, etc., used as food products, and is hence highly safe to the human body, and the like. Also, as to be described later in Examples, the immunostimulant can exhibit immunostimulating actions even in a low concentration. Consequently, according to the present embodiment, an immunostimulant having immunostimulating actions in a small amount with no or extremely scarce side effects can be provided.

### 2. Pharmaceutical Composition

The second embodiment of the present invention is a pharmaceutical composition. The pharmaceutical composition of the present invention is a drug to stimulate the immunity in vivo and aims to prevent and/or treat a disease by enhancing the immunological effect. The pharmaceutical composition of the present invention is characterized by containing the immunostimulant of the above first embodiment as the active ingredient.

In the present specification, the “prevention” refers to the prevention of a disease incidence. The “treatment” refers to alleviate or remove a disease infected and/or conditions associated therewith.

### 2-1. Constitution

The pharmaceutical composition of the present invention may contain, within the range wherein the immunostimulating actions of the immunostimulant of the first embodiment are not inhibited or suppressed, a pharmaceutically acceptable carrier and/or drugs having the same or different pharmacological efficacies such as gastrointestinal drugs, etc.

### 2-1-1. Pharmaceutically Acceptable Carrier

Examples of the “pharmaceutically acceptable carrier” include pharmaceutically acceptable excipients, binders, disintegrators, fillers, emulsifiers, flow modifiers and lubricants.

Examples of the excipient include sugars such as monosaccharides, disaccharides, cyclodextrin and polysaccharides (specifically, but not limited to, include glucose, sucrose, lactose, raffinose, mannitol, sorbitol, inositol, dextrin, maltodextrin, starch and cellulose), metal salts (e.g., sodium phosphate or calcium phosphate, calcium sulfate, magnesium sulfate), citric acid, tartaric acid, glycine, low-, medium-, high molecular weight polyethylene glycol (PEG), pluronic and combinations thereof.

Examples of the binder include starch pastes using starches of corn, wheat, rice, or potato, gelatin, tragacanth, methylcellulose, hydroxypropylmethylcellulose, carboxymethyl cellulose sodium and/or polyvinylpyrrolidone.

Examples of the disintegrator include the above mentioned starches, carboxymethyl starch, crosslinked polyvinylpyrrolidone, agar, alginic acid or sodium arginine and salts thereof.

Examples of the filler include the above sugars and/or calcium phosphate (e.g., tricalcium phosphate or dibasic calcium phosphate).

Examples of the emulsifier include sorbitan fatty acid ester, glycerine fatty acid ester, sucrose fatty acid ester and propylene glycol fatty acid ester.

Examples of the flow modifier and the lubricant include silicate, talc, stearate and polyethylene glycol.

These carriers allow easy formulation of the pharmaceutical composition and are used to maintain the drug efficacies as the immunostimulant of the present invention contained therein, and may suitably be used as necessary. The pharmaceutical composition of the present invention may further contain, as necessary, in addition to the above additives, corrigents, solubilizing adjuvants (solubilizers), suspension agents, diluents, surfactants, stabilizers, absorption enhancers, (e.g., quarternary ammonium salts, sodium lauryl sulfate, etc.), extenders, humidifiers, moisturizers (e.g., glycerol, starches, etc.), sorbents (e.g., starches, lactose, kaoline, bentonite, colloidal silicic acid, etc.), disintegrators (e.g., white sugar, stearin, cacao butter, hydrogenated oil, etc.), coating agents, coloring agents, preservatives, antioxidants, perfumes, flavoring agents, sweeteners, buffers, etc.

### 2-1-2. Prodrug

The pharmaceutical composition of the present invention may also contain BR, which is the active ingredient, contained in the immunostimulant of the first embodiment or a pharmaceutically acceptable salt thereof in the form of a prodrug. The “prodrug” is a compound which easily undergoes a chemical conversion under physiological conditions and, as a result, provides an active form of an intended pharmacological action. For example, the prodrug is a compound present in the pharmaceutical composition as that having a different structure from that of BR before administration and is converted to BR or an active form thereof by, for example, the action of digestive enzymes in the gastrointestinal tract after administration. Further, the prodrug can encompass a compound which is converted in the ex vivo environment to BR or an active form thereof by a chemical or biochemical method.

### 2-1-3. Content of Immunostimulant

The content of the immunostimulant of the first embodiment contained in the pharmaceutical composition of the present invention varies depending on the type and/or effective amount of the immunostimulant to be used, the dosage form (including formulation and size) of the pharmaceutical composition and the type of a carrier to be added, and is suitably determined based on each condition.

An example of the content may be, when the pharmaceutical composition of the present invention is formulated as a solid preparation, 0.00001 to 50 wt. %, preferably 0.00001 to 30 wt. %, more preferably 0.00001 to 20 wt. %, of the immunostimulant of the first embodiment per tablet. More specifically, when preparing a tablet weighing 200 mg, each tablet may typically contain 2 μg to 100 mg of BR. Other than the immunostimulant, the above additives can be contained as the pharmaceutically acceptable carriers in the content rate known in the art.

However, in the solid preparation, the effective amount of the immunostimulant can be adjustable by the number of tablets and, for this reason, the effective amount does not necessarily have to be contained in a tablet but per administration unit, that is, when several tablets are taken, the effective amount of the immunostimulant may be contained in total per administration unit.

The “effective amount” used herein in the present specification refers to the amount required for BR or a pharmaceutically acceptable salt thereof to exhibit the function as the active ingredient, that is, in the present invention, the amount required for the immunostimulant to impart immuno stimulating actions to the living body and almost free of or no adverse side effects to a patient to whom it is administered. The effective amount is variable depending on various conditions such as information on a subject and administration route, etc.

The “information on a subject administered” includes general health conditions, progress and severity of a disease, age, weight, sex, diet, drug sensitivity, concomitant drug if any, tolerance against treatment, and the like. The final effective amount of the above immunostimulant and the dose calculated based on the amount are determined in the end by the decision made by a doctor or a veterinarian in accordance with the information on an individual subject to be administered (a patient if a subject is a human), and the like.

When the pharmaceutical composition of the present invention is prepared as an injection, other examples of the content of the immunostimulant contained in the injection per administration unit are typically 0.1 to 30% (w/v), preferably 0.1 to 20% (w/v), more preferably 0.1 to 10% (w/v). More specifically, for example, when an injection weighing 2 mL per administration is prepared, the injection can typically contain 2 to 600 μg of BR.

### 2-1-4. Dosage Form of Pharmaceutical Composition

The dosage form of the pharmaceutical composition of the present invention varies depending on dosage and/or prescription condition. The administration method can usually be classified into oral administration and parenteral administration. These administrations are to be described later.

Examples of the dosage form suitable for oral administration include solid preparations (including tablets, pills, sublingual tablets, capsules, drops, troches), granules, powders, pulvis, and solutions, and the like. The solid preparations can further be formulated as necessary into dosage form treated with coatings known in the art, such as sugar-coated tablets, gelatin coated tablets, enteric coated tablets, film coated tablets, double layer tablets and multilayer tablets.

The parenteral administration is further classified into interstitial administration, local administration and transrectal administration, and the pharmaceutical composition of the present invention can also be formulated into a dosage form suitable for each dosage. An example of the dosage form suitable for interstitial administration includes an injection, which is a solution. Example of the dosage form suitable for local administration include solutions (liniments, ophthalmic solutions, nasal drops, inhalants), suspensions (including emulsions, creams), powders (including nasal powder, inhalant powder), pastes, gels, ointments, plasters, and the like. Examples of the dosage form suitable for transrectal administration include suppositories, and the like.

The pharmaceutical composition of the present invention is preferably formulated into a dosage form suitable for oral administration due to low invasiveness and easy administration since BR has a low molecular weight and high intestinal absorption property.

The specific shape and size of each of the above dosage forms are not limited insofar as they are within the range wherein each of the dosage form is formulated into the dosage form known in the art.

### 2-2. Preparation of Pharmaceutical Composition

To formulate the pharmaceutical composition of the present invention, a method known in the art can be used in principle. For example, the method described in Remington's Pharmaceutical Sciences (Merck Publishing Co., Easton, Pa.) can be used.

For example, an injection can be prepared by the method routinely practiced in the art using the immunostimulant of the above first embodiment dissolved in a pharmaceutically acceptable solvent.

Examples of the pharmaceutically acceptable solvent include water, ethanol, propylene glycol, ethoxylated isostearyl alcohol, polyoxylated isostearyl alcohol, and polyoxyethylene sorbitan fatty acid esters. They are desirably sterilized and, as necessary, adjusted to be isotonic with blood.

### 2-3. Dosage of Pharmaceutical Composition

The dosage of the pharmaceutical composition of the present invention can be administered in the known dosage unit form in the art. Examples of the dosage unit form include oral administration, interstitial administration (e.g., subcutaneous administration, intramuscular administration, intravenous administration, etc.), local administrations (e.g., dermal administration, etc.), transrectal administration, and the like. The pharmaceutical composition of the present invention may use any of these dosage unit forms. However, the oral administration is preferable due to the low invasiveness and easy administration since BR has a low molecular weight and high intestinal absorption property as described earlier.

On the other hand, a preferable dosage for the interstitial administration of the pharmaceutical composition of the present invention is injection.

For injection, the injection site is not limited. Examples of the injection site include intravenous, intraarterial, intrahepatic, intramuscular, intraarticular, intra-bone marrow, intraspinal, intraventricular, percutaneous, subcutaneous, intradermal, intraperitoneal, intranasal, intraintestinal, sublingual sites and the like. Preferable injections are intravascular injections such as intravenous injection and intraarterial injection. Such a dosage allows the immunostimulant, the active ingredient of the present invention to be immediately carried throughout the entire body via the blood flow and also has comparatively low invasiveness with minimal burden to a subject to be administered.

For the administration of the pharmaceutical composition of the present invention, it is desirable that the effective amount capable of exhibiting the immuno stimulating actions is contained in a single dosage unit as described above.

A specific example of the dose ranges, when orally administered to a human adult who does not require other concomitant drugs, typically from 0.1 to 1000 mg/kg of body weight, preferably from 1 to 100 mg/kg of body weight as the effective amount of the immunostimulant per day. In accordance with the conditions of a patient or administration route, etc., a dose below the above range or exceeding the above range can also be administered. When a large dose of the above pharmaceutical composition is required to be administered to attain the immunostimulating effect, the composition can be administered in several divided doses to reduce the burden to a subject.

### 3. Drink and Food Products for Immunostimulation

The third embodiment of the present invention is food and drink products for immunostimulation. The food and drink products for immunostimulation of the present invention are those stimulating the immunological effects of an animal that consumed the products, and are characterized by containing an effective amount of the immunostimulant of the first embodiment as the active ingredient.

### 3-1. Constitution

The “drink and food products for immunostimulation” of the present invention are those aiming at stimulating the immunological effects through drinking and eating. The “drink and food products” used herein encompass not only the drinks, food and/or health food products consumed by human but also fodders, livestock feeds, and diets (including pet foods) fed to livestock (including cultured fish), racehorse, companion animals, animals for admiration, and the like.

The form of these drink and food products is not limited. For example, the form may be preparations, processed food products or various beverages.

### 3-1-1. Drink and Food Products for Immunostimulation in the Form of Preparation

The “drink and food products for immunostimulation” of the present invention, when formulated into a preparation such as health foods, functional foods or foods for specified health uses as the composition of the drink and food products for immunostimulation, can be constituted according to the pharmaceutical composition of the second embodiment. For example, in place of the pharmaceutically acceptable carrier, a food acceptable carrier may be contained. Examples of the “food acceptable carrier” used herein include food additives, and the like, regulated in Food Sanitation Act. Further, the content of the immunostimulant in the drink and food products for immunostimulation can be those according to the pharmaceutical composition of the second embodiment. Furthermore, the dosage form also can be those according to the pharmaceutical composition of the second embodiment, e.g., tablets, chewable tablets, powders, capsules, granules and health drinks.

### 3-1-2. Drink and Food Products for Immunostimulation in the Form of Processed Food Product

To form the “drink and food products for immunostimulation” of the present invention into a processed food product, the effective amount of the immunostimulant of the first embodiment can be added. Examples of the processed food product include breads, noodles, spreads (including butter, margarine, jams, furikake sprinkles, dressings, mayonnaise, etc.), pastas, miso soybean pastes, tofu, milk, yogurt, soups or sauces, snacks (e.g., biscuits, cookies, chocolates, candies, cakes, ice creams, chewing gums, tablets), etc. Similarly, for fodders, livestock feeds and diets, the effective amount of the immunostimulant of the first embodiment may be added to known fodders, livestock feeds and diets.

To prepare the “drink and food products for immunostimulation” of the present invention as a beverage, the effective amount of the immunostimulant of the first embodiment can similarly be added to the known beverages. Examples of the beverage include teas such as green tea, oolong tea, English teas, and the like, soft drinks, jelly drinks, sport drinks, milk drinks, carbonated drinks, fruit juice drinks, lactic acid bacteria drinks, fermented milk drinks, powder drinks, cocoa drinks, purified water, etc.

The effective amount of the immunostimulant of the first embodiment to be added to the drink and food products can be an amount according to the pharmaceutical composition of the second embodiment. The content of the first embodiment in the drink and food products for immunostimulation varies depending on the form, but, on the dry mass basis, ranges typically from 0.001 to 80 mass %, preferably 0.01 to 50 mass %, more preferably 1 to 50 mass %. To consume the above described intake per day for an adult, it is desirable that the product be in the shape due to which the daily intake can be controlled.

### 3-2. Manufacture of the Drink and Food Products for Immunostimulation

The manufacture of the drink and food products for immunostimulation can be achieved by adding the immunostimulant of the first embodiment in suitable steps basically in accordance with the method for manufacturing the product intended. For example, the drink and food products in the form of preparation can be manufactured in the same manner as that for the pharmaceutical composition of the second embodiment. The dosage, if orally administered, can basically follow the pharmaceutical composition of the second embodiment. The processed food products and various drinks, etc., are manufactured in accordance with the known manufacturing processes of the product intended. For the manufacture of the drink and food products for immunostimulation, other food materials, nutrients, vitamins, minerals, amino acids and oils and fats used for manufacturing various food products can be added in accordance with a routine method. Further, the immunostimulant of the present invention can be added to the commonly consumed food products such as rice.

### 3-3. Effect

The drink and food products of the present invention are highly safe and can be orally consumed continuously over a long period of time on a daily basis since the products contain the immunostimulant of the first embodiment as the active ingredient. The routine consumption can stimulate the immune system of an individual and consequently prevent and/or alleviate various diseases such as infectious diseases to begin with.

### Example

### Example 1

### Effect of BL on B Cell Proliferation by Antigen Stimulation

The effect of BL on the spleen cell proliferation caused by an antigen stimulation was examined.

### (Materials and Methods)

A mixed solution (0.2 mL) of 10 g of ovalbumin (Sigma-Aldrich Corporation) and 4 mg of aluminum hydroxide (Thermo Scientific Pierce Protein Research Products) was intraperitoneally administered to a BALB/c mouse (female, 8 to 12 weeks old) and the spleen was collected 2 weeks later. Cells were isolated from the spleen and cultured in RPMI-1640 medium containing 10% fetal bovine serum (Cansera International Inc.), 5×10⁻⁵M β-mercaptoethanol (Wako Pure Chemical Industries, Ltd.), 20 U/mL penicillin (Meiji Seika Kaisha, Limited) and 100 μg/mL streptomycin (Meiji Seika Kaisha, Limited) at 37° C. under the presence of 5% CO₂. Cells to which BL (Brassino Co., Ltd.) was added in a concentration of 10 nM and cells to which BL was not added were respectively stimulated by an antigen (10 g/mL of ovalbumin), cultured for 3 days and measured for the incorporated BrdU in the detection of proliferating spleen cells using a cell proliferation ELISA BrdU colorimetric kit (Roche Applied Science).

### (Results)

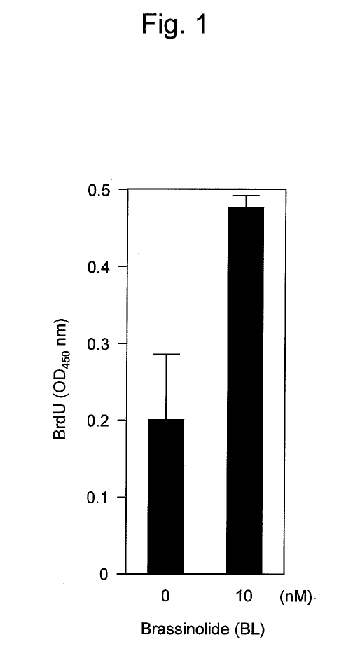

The results are shown in FIG. 1. The spleen cell proliferation induced by the antigen stimulation was significantly promoted by the addition of BL in an amount as small as 10 nM. Usually, about 50% of the spleen cells is B cells and about 40% is T cells. Consequently, BL is verified to activate the proliferation of B cells and T cells contributing to the acquired immune system, in other words, BL is verified to have the immunostimulating action.

### Example 2

### Effect of BL on the Antibody Production in Mouse

### (Materials and Methods)

A mixed solution (0.2 mL) of 10 g of ovalbumin (Sigma-Aldrich Corporation) and 4 mg of aluminum hydroxide (Thermo Scientific Pierce Protein Research Products) was intraperitoneally administered to a BALB/c mouse (female, 8 to 12 weeks old) and BL dissolved in 0.2 mL PBS (0, 0.2, 1 and 5 mg/Kg of body weight) was intraperitoneally administered every other day. The serum was isolated 2 weeks later and measured for the production of immunoglobulin (IgG) specific to the antigen (ovalbumin) using ELISA. ELISA was carried out using a biotinylated anti-mouse IgG antibody (Southern Biotechnology Associates Inc.) as the secondary antibody, streptavidin-HRP (Prozyme, Inc.) and a TMB Microwell Peroxidase Substrate (Kirkegaard & Perry Laboratories, Inc.) as the colorimetric substrate, and the absorbance at 450 nm was measured.

### (Results)

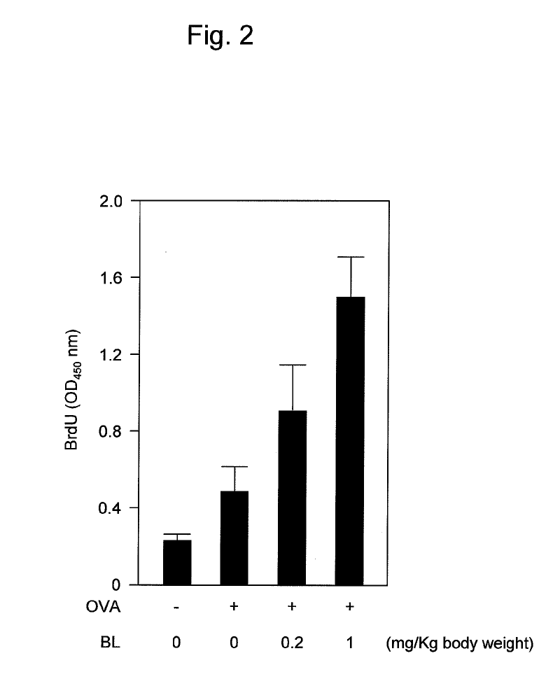

The results are shown in FIG. 2. The antigen specific antibody productions induced by the stimulations of ovalbumin and aluminum hydroxide were significantly promoted by the addition of BL in a dose-dependent manner.

### Example 3

### Effect BL on Innate Immune Response

The effect of BL on the innate immune response was verified.

### (Materials and Methods)

3 mL of 3% thioglycolate medium (Difco Laboratories) was intraperitoneally administered to a BALB/c mouse (female, 8 to 12 weeks age), peritoneal cells were collected 3 days later and cultured in RPMI-1640 medium containing 10% fetal bovine serum (Cansera International Inc.), 5×10⁻⁵M β-mercaptoethanol (Wako Pure Chemical Industries, Ltd.), 20 U/mL penicillin (Meiji Seika Kaisha, Limited) and 100 μg/mL streptomycin (Meiji Seika Kaisha, Limited) at 37° C. under the presence of 5% CO₂. After culturing 1×10⁶/mL of the peritoneal cells for 3 hours in a 6-well plate (2 mL/well), non-adherent cells were removed and adherent cells were tested as the macrophages. The macrophages were cultured for 15 minutes in the presence or absence of 10 nM BL, subsequently stimulated with R848 (InvivoGen) or LPS (Sigma-Aldrich Corporation) 100 ng/mL, and the RNA was collected 1 hour later using Rneasy Mini Kit (QIAGEN K. K.). Using a Primescript RT reagent kit (TAKARA BIO INC.), cDNA was synthesized from 1 g of the isolated RNA in accordance with the attached protocol and the real time PCR was carried out using Thermal Cycler Dice Real Time System II (TAKARA BIO INC.) to examine the changes of cytokine (IFN-α, IFN-β, IL-12 subunit p35, IL-12 subunit p40, TNF-α) mRNA expression. The cytokine specific primers used in the real time PCR are as shown in Table 1.

TABLE 1

| Transcription product | F/R | Site | Sequence | Sequence No. | Accession # |
|---|---|---|---|---|---|
| HPRT | F | 209-228 | GGGGCTATAAGTTCTTTGCT | SEQ ID NO. 1 | NM_013556 |
| | R | 352-371 | GTTGAGAGATCATCTCCACC | SEQ ID NO. 2 | |
| IL-12 p35 | F | 575-594 | AGACTCTGCGCCAGAAACCT | SEQ ID NO. 3 | NM_001159424.1 |
| | R | 684-703 | AGCTCAGATAGCCCATCACC | SEQ ID NO. 4 | |
| IL-12 p40 | F | 409-428 | TTCACGTGCTCATGGCTGGT | SEQ ID NO. 5 | NM_008352 |
| | R | 532-551 | TAGTCCCTTTGGTCCAGTGT | SEQ ID NO. 6 | |
| TNF-α | F | 290-309 | CAAACCACCAAGTGGAGGAG | SEQ ID NO. 7 | NM_013693 |
| | R | 425-444 | GCAGCCTTGTCCCTTGAAGA | SEQ ID NO. 8 | |
| IFN-α (universal) | F | 378-399 | GGAACCTCCCCTGACCCAGGAA | SEQ ID NO. 9 | NM_010502.2 |
| | R | 493-512 | AGGGCTCTCCAGACTTCTGCTC | SEQ ID NO. 10 | |
| INF-β | F | 177-196 | CCCTATGGAGATGACGGAGA | SEQ ID NO. 11 | NM_010510.1 |
| | R | 322-341 | AACACTGTCTGCTGGTGGAG | SEQ ID NO. 12 | |

F: forward primer; R: reverse primer

### (Results)

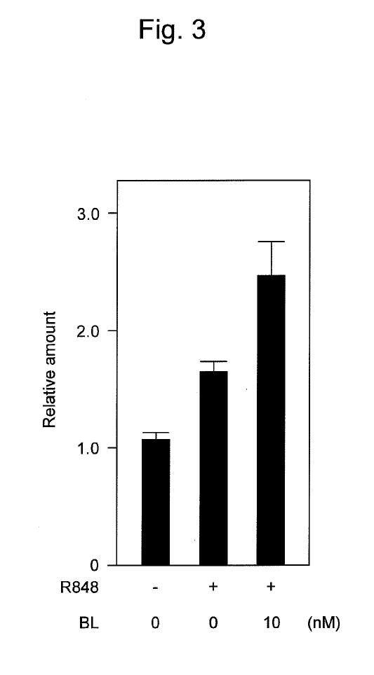

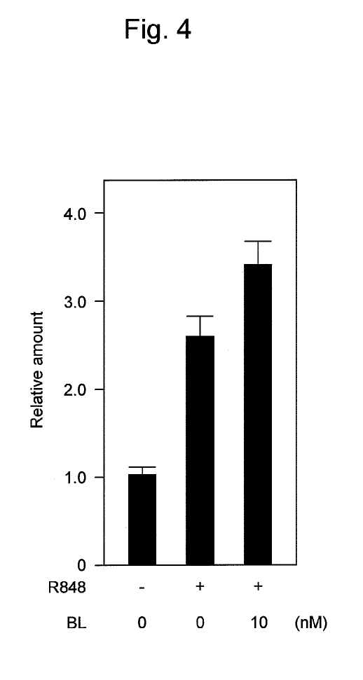

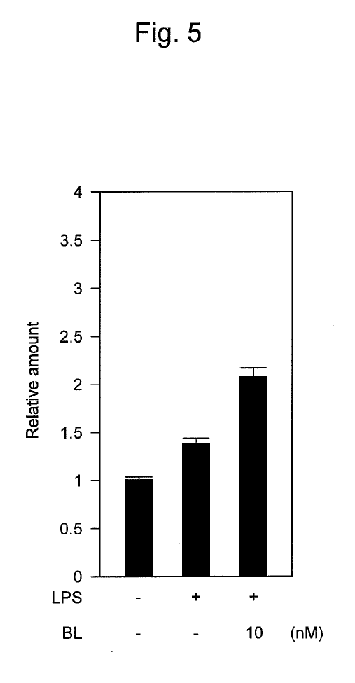

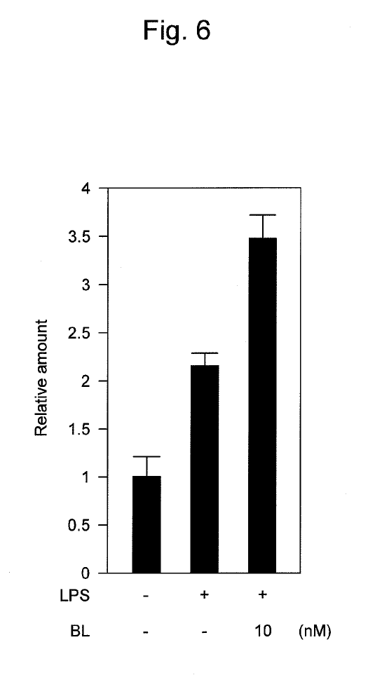

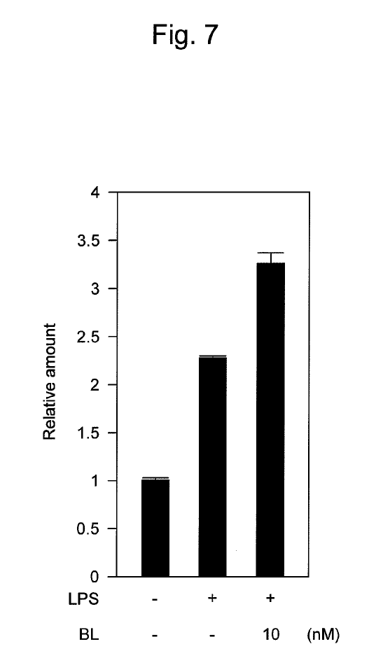

The results are shown in FIGS. 3 to 7. The mRNA amounts are shown for IFN-α in FIG. 3, IFN-β in FIG. 4, IL-12 subunit p35 in FIG. 5, IL-12 subunit p40 in FIG. 6 and TNF-α in FIG. 7. The mRNA amount in each Figure is indicated as the relative amount to the mRNA amount of HPRT, which is the housekeeping gene.

As evident in FIGS. 3 to 7, it is revealed that BL, in a concentration as low as 10 nM, is capable of enhancing the mRNA expression induction for IFN-α and IFN-β by the activation of TRL-7 induced by the R848 stimulation and the mRNA expression induction for IL-12 p35, IL-12 p40 and TNF-α by the activation of TRL-4 induced by the LPS stimulation.

All publications, patents and patent applications cited herein shall be incorporated per se by references in the specification.

1. An immunostimulant comprising as an active ingredient a compound represented by the following general formula (I) or a pharmaceutically acceptable salt thereof. 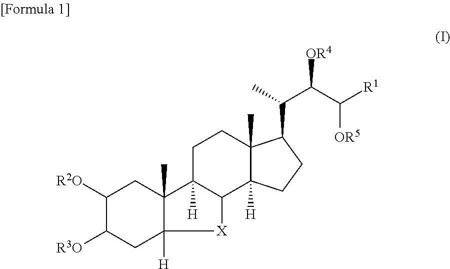
wherein,
R¹ is a hydrogen atom or a substituted or unsubstituted alkyl group having 1 to 6 carbon atoms,
R² to R⁵ are independently a hydrogen atom, or a substituted or unsubstituted lower alkyl or lower acyl group,
X is —(CH₂)₂—, —(CH₂)₃—, —(CH₂)₄—, —CO—CH₂—, —CO—(CH₂)₂—, —CO—(CH₂)₃—, —CH₂—CO—CH₂—, —CO—O—CH₂—, —CO—O—(CH₂)₂—, —O—CO—CH₂—, —O—CO—(CH₂)₂—, —CH₂—O—CH₂—, —CH₂—O—(CH₂)₂—, —O—CH₂—, —O—(CH₂)₂—, —O—(CH₂)₃—, —NHY—CO—CH₂—, —NHYCO—(CH₂)₂—, —CO—NHY—CH₂— or —CO—NHY—(CH₂)₂—,
Y herein is a hydrogen atom or a lower alkyl group.

2. The immunostimulant according to claim 1 wherein R¹ is a 1,2-dimethylpropyl group.

3. The immunostimulant according to claim 1 wherein R² to R⁵ are a hydrogen atom.

4. The immunostimulant according to claim 1 wherein X is —CO—O—CH₂—.

5. The immunostimulant according to claim 4 represented by the following formula (II).

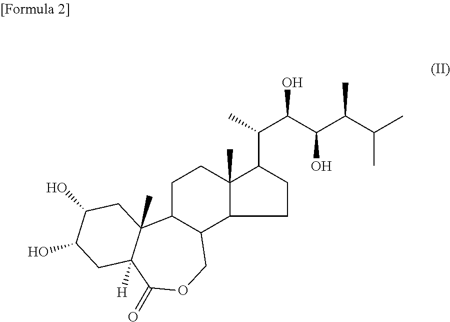

6. A pharmaceutical composition comprising the immunostimulant according to claim 1 as an active ingredient.

7. A food or drink product for immunostimulation comprising an immunostimulant comprising as an active ingredient a compound represented by the following general formula (I) or a pharmaceutically acceptable salt thereof. 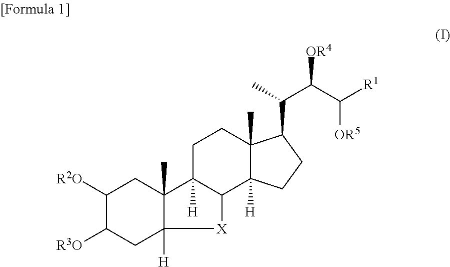
wherein,
R¹ is a hydrogen atom or a substituted or unsubstituted alkyl group having 1 to 6 carbon atoms,
R² to R⁵ are independently a hydrogen atom, or a substituted or unsubstituted lower alkyl or lower acyl group,
X is —(CH₂)₂—, —(CH₂)₃—, —(CH₂)₄—, —CO—CH₂—, —CO—(CH₂)₂—, —CO—(CH₂)₃—, —CH₂—CO—CH₂—, —CO—O—CH₂—, —CO—O—(CH₂)₂—, —O—CO—CH₂—, —O—CO—(CH₂)₂—, —CH₂—O—CH₂—, —CH₂—O—(CH₂)₂—, —O—CH₂—, —O—(CH₂)₂—, —O—(CH₂)₃—, —NHY—CO—CH₂—, —NHYCO—(CH₂)₂—, —CO—NHY—CH₂— or —CO—NHY—(CH₂)₂—,
Y herein is a hydrogen atom or a lower alkyl group as an active ingredient.


SEQUENCE LISTING (12 sequences)

SEQ ID NO: 1 - DNA, 20 bp, Artificial
  primer
ggggctataagttctttgct

SEQ ID NO: 2 - DNA, 20 bp, Artificial
  primer
gttgagagatcatctccacc

SEQ ID NO: 3 - DNA, 20 bp, Artificial
  primer
agactctgcgccagaaacct

SEQ ID NO: 4 - DNA, 20 bp, Artificial
  primer
agctcagatagcccatcacc

SEQ ID NO: 5 - DNA, 20 bp, Artificial
  primer
ttcacgtgctcatggctggt

SEQ ID NO: 6 - DNA, 20 bp, Artificial
  primer
tagtccctttggtccagtgt

SEQ ID NO: 7 - DNA, 20 bp, Artificial
  primer
caaaccaccaagtggaggag

SEQ ID NO: 8 - DNA, 20 bp, Artificial
  primer
gcagccttgtcccttgaaga

SEQ ID NO: 9 - DNA, 22 bp, Artificial
  primer
ggaacctcccctgacccaggaa

SEQ ID NO: 10 - DNA, 22 bp, Artificial
  primer
agggctctccagacttctgctc

SEQ ID NO: 11 - DNA, 20 bp, Artificial
  primer
ccctatggagatgacggaga

SEQ ID NO: 12 - DNA, 20 bp, Artificial
  primer
aacactgtctgctggtggag